(19) United States
(12) Patent Application Publication
(10) Pub. No.: US 2006/0210600 A1
(43) Pub. Date: Sep. 21, 2006

(54) Coated stent with timed release of multiple therapeutic agents to inhibit restenosis adjacent to the stent ends

(75) Inventor: John Nolting, Poway, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/563,785

(22) PCT Filed: Jul. 1, 2004
(86) PCT No.: PCT/US04/21506

§ 371 Date: Apr. 25, 2006

Related U.S. Application Data
(60) Provisional application No. 60/485,154, filed on Jul. 7, 2003.

(51) Int. Cl. A61F 2/00

(52) U.S. Cl. 424/423; 623/1.42; 427/2.1

(57) ABSTRACT

The present invention provides a system for treating a vascular condition that inhibits restenosis adjacent to the ends of a stent by delivering a therapeutic agent to the vessel wall at and beyond the stent ends. The system comprises a catheter and a coated stent operably coupled to the catheter. The coated stent includes a plurality of therapeutic coatings disposed on the distal and proximal ends of a stent framework. A plurality of therapeutic agents is released from the plurality of therapeutic coatings, the therapeutic agents being released sequentially to inhibit restenosis adjacent to the ends of the stent. The system may further include a therapeutic agent and a timing coating disposed on a mid-portion of the stent framework.

### TECHNICAL FIELD

This invention relates generally to biomedical devices that are used for treating vascular conditions. More specifically, the invention relates to a coated stent that provides timed release of multiple therapeutic agents that are positioned at the ends of the stent to inhibit restenosis at the stent ends.

### BACKGROUND OF THE INVENTION

Stents are generally cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the diameter of the stent to expand. For a self-expanding stent, commonly a sheath is retracted, allowing expansion of the stent.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow.

Soon after the procedure, however, a significant proportion of treated vessels restenose. To prevent restenosis, a stent, constructed of a metal or polymer, is implanted within the vessel to maintain lumen size. The stent acts as a scaffold to support the lumen in an open position. Configurations of stents include a cylindrical tube defined by a solid wall, a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Stent insertion may cause undesirable reactions such as inflammation, infection, thrombosis, and proliferation of cell growth that occludes the passageway. Therapeutic agents that assist in preventing these conditions have been delivered to the site by coating these agents onto a stent. Restenosis is often a greater problem adjacent to the ends of a stent than it is elsewhere along the stent. This greater problem is not addressed by prior art stents that carry the same therapeutic agent at the same dose throughout the stent. The problem is also not fully addressed by prior art stents that carry only a single therapeutic agent concentrated on the ends of the stent or that carry multiple therapeutic agents that are not tailored for release at a predetermined time. Restenosis is a disease state that expresses itself differently as the disease progresses and elicits varied responses from the body's immune system at different stages of the disease. Certain therapeutic agents are most effective when released during a specific stage of the disease.

Therefore, it would be desirable to have a coated stent, a system for treating a vascular condition, and a method of inhibiting restenosis adjacent to the ends of a stent used to treat a vascular condition that overcome the aforementioned and other disadvantages.

### SUMMARY OF THE INVENTION

One aspect of the present invention is a system for treating a vascular condition, comprising a catheter and a coated stent operably coupled to the catheter. The coated stent includes a plurality of therapeutic coatings disposed on the distal and proximal ends of the stent. A plurality of therapeutic agents is released from the plurality of therapeutic coatings, the therapeutic agents being released sequentially to inhibit restenosis adjacent to the ends of the stent.

Another aspect of the present invention is a coated stent. The coated stent comprises a stent framework and a plurality of therapeutic coatings disposed on the distal and proximal ends of the stent framework. A plurality of therapeutic agents is released from the plurality of therapeutic coatings, the therapeutic agents being released sequentially to inhibit restenosis adjacent to the ends of the stent.

Yet another aspect of the present invention is a method of inhibiting restenosis adjacent to the ends of a stent used to treat a vascular condition. A coated stent is provided. The coated stent includes a first and a second therapeutic coating disposed on the distal and proximal ends of a stent framework. The first therapeutic coating includes a first therapeutic agent. The second therapeutic coating includes a second therapeutic agent. The coated stent further includes a first timing coating positioned between the first and second therapeutic coatings. The first therapeutic agent is released from the first therapeutic coating. The first timing coating is actuated. The second therapeutic agent is released from the second therapeutic coating at a time controlled by the first timing coating.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a system for treating a vascular condition, in accordance with the present invention;

FIG. 2 is an enlarged, fragmentary view of a coated stent in accordance with the present invention;

FIG. 3 is a graphic representation of the release of therapeutic agents from the coated stent of FIG. 2; and

FIG. 4 is a flow diagram of one embodiment of a method of inhibiting restenosis adjacent to the ends of a stent used to treat a vascular condition, in accordance with the present invention.

### DETAILED DESCRIPTION OF THE PRESENTLY PREFERRED EMBODIMENTS

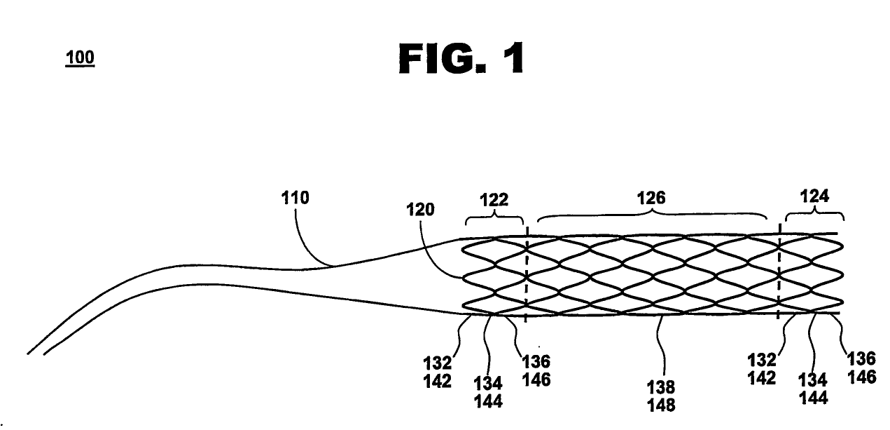

One aspect of the present invention is a system for treating a vascular condition. One embodiment of the system, in accordance with the present invention, is illustrated in FIG. 1 at 100. System 100 comprises a catheter 110 and a coated stent 120. Coated stent 120 comprises a proximal end 122, a distal end 124, and a mid-portion 126. Coated stent 120 includes therapeutic coatings 132,134,136, and 138. Therapeutic coatings 132, 134, and 136 are disposed on the proximal and distal ends of the stent. Therapeutic coating 138 is disposed on the mid-portion of the stent. Coated stent 120 further includes timing coatings 142, 144, 146, and 148. Timing coatings 142, 144, and 146 are disposed on the proximal and distal ends of the stent, alternating with therapeutic coatings 132, 134, and 136. Timing coating 148 is disposed on the mid-portion of the stent.

Catheter 110 may be any catheter known in the art that is appropriate for delivering a stent to a treatment site, for example a percutaneous transluminal coronary angioplasty (PTCA) balloon catheter.

Coated stent 120 is operably coupled to catheter 110. Coated stent 120 may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

Coated stent 120 includes therapeutic coatings 132, 134, and 136, indicated generally in FIG. 1, disposed on the proximal 122 and distal 124 ends of the stent. While the present embodiment includes three therapeutic coatings, one skilled in the art will recognize that a coated stent in accordance with the invention may include more coatings or may include just two coatings. The therapeutic coatings may comprise a bioerodable polymer and a therapeutic agent. The therapeutic agents released from these coatings may be, for example, an antiproliferative agent, an antineoplastic agent, an antibiotic agent, an anti-inflammatory agent, a free radical scavenger, a protein, combinations thereof, and the like. More specifically, the therapeutic agents may be paclitaxel, dexamethasone, rapamycin, a rapamycin analog, a nonsteroidal anti-inflammatory drug, a steroidal anti-inflammatory drug, a superoxide dismutase mimic, apo A-1 Milano, combinations thereof, and the like. Each coating may release a different therapeutic agent, or the same agent may be included in more than one coating.

Stent 120 further includes timing coatings 142, 144, and 146, indicated generally in FIG. 1, disposed on the proximal 122 and distal 124 ends of the stent. One skilled in the art will recognize that a coated stent in accordance with the present invention may include more or fewer timing coatings than the three indicated in FIG. 1. The timing coatings may comprise a bioerodable polymer. Timing coatings 142, 144, and 146 alternate with therapeutic coatings 132, 134, and 136, preventing release of the therapeutic agent positioned beneath the timing coating until a predetermined time. The time of release may be controlled by characteristics of the timing coating such as the timing coating's thickness, its permeability, and its resistance to being hydrolyzed and thus eroded, and other such characteristics.

In the present embodiment, therapeutic coating 136 is positioned nearest the stent, with timing coating 146 positioned over therapeutic coating 136 to control the time at which the therapeutic agent is released from therapeutic coating 136. Therapeutic coating 134 is positioned over timing coating 146 and is controlled by timing coating 144. Therapeutic coating 132 is positioned over timing coating 144 and is controlled by timing coating 142, which is the outermost of the coatings. The therapeutic and timing coatings are selected and positioned to release the therapeutic agents at the appropriate times and for the appropriate durations to most effectively inhibit restenosis adjacent to the ends of the stent.

In the present embodiment, coated stent 120 additionally includes a therapeutic coating 138 disposed on the mid-portion of the stent. A coated stent in accordance with the present invention may, however, include coatings on only the ends of the stent. Therapeutic coating 138 may release a therapeutic agent that is different from the therapeutic agents released from therapeutic coatings 132, 134, and 136, or it may display diffusion characteristics that are different from those of coatings 132, 134, and 136. Timing coating 148 controls the time at which therapeutic coating 138 begins to release its therapeutic agent.

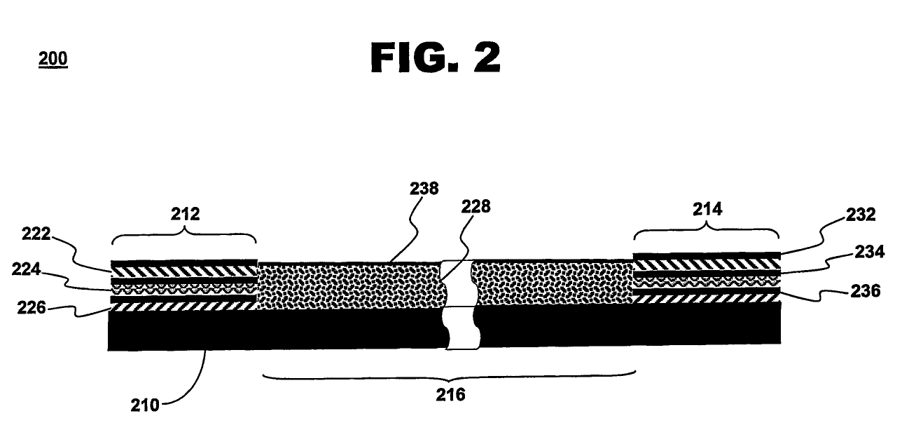

Another aspect of the present invention is a coated stent. FIG. 2 at 200 shows an enlarged, fragmentary view of one embodiment of the coated stent, in accordance with the present invention. Coated stent 200 comprises a stent framework 210 having a proximal end 212, a distal end 214, and a mid-portion 216. Coated stent 200 includes therapeutic coatings 222, 224, 226, and 228. Therapeutic coatings 222, 224, and 226 are disposed on the proximal and distal ends of the stent framework. Therapeutic coating 228 is disposed on a mid-portion of the stent framework. Coated stent 200 further includes timing coatings 232, 234, 236, and 238. Timing coatings 232, 234, and 236 are disposed on the proximal and distal ends of the stent framework, alternating with therapeutic coatings 222, 224, and 226. Timing coating 238 is disposed on the mid-portion of the stent.

Stent framework 210 may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

Therapeutic coatings 222, 224, and 226, disposed on the proximal 212 and distal 214 ends of stent framework 210, may comprise a bioerodable polymer and a therapeutic agent. The therapeutic coatings may each release a different therapeutic agent, or the same agent may be included in more than one coating. The therapeutic agents may be, for example, an antiproliferative agent, an antineoplastic agent, an antibiotic agent, an anti-inflammatory agent, a free radical scavenger, a protein, combinations thereof, and the like. More specifically, the therapeutic agents may be paclitaxel, dexamethasone, rapamycin, a rapamycin analog, a nonsteroidal anti-inflammatory drug, a steroidal anti-inflammatory drug, a superoxide dismutase mimic, apo A-1 Milano, combinations thereof, and the like. While the present embodiment includes three therapeutic coatings, one skilled in the art will recognize that a coated stent In accordance with the invention may include more coatings or may include just two coatings.

Coated stent 200 further includes timing coatings 232, 234, and 236 disposed on the proximal 212 and distal 214 ends of stent framework 210. These timing coatings may comprise a bioerodable polymer. Timing coatings 232, 234, and 236 alternate with therapeutic coatings 222, 224, and 226, preventing release of the therapeutic agent positioned beneath the timing coating until a predetermined time. The time of release may be controlled by characteristics of the timing coating such as the timing coating's thickness, its permeability, its resistance to being hydrolyzed and thus eroded, and other such characteristics.

As shown in FIG. 2, therapeutic coating 226 is positioned nearest stent framework 210, with timing coating 236 positioned over therapeutic coating 226 to control the time at which the therapeutic agent is released from therapeutic coating 226. Therapeutic coating 224 is positioned over timing coating 236 and is controlled by timing coating 234. Therapeutic coating 222 is positioned over timing coating 234 and is controlled by timing coating 232, which is the outermost of the coatings.

The therapeutic and timing coatings are intended to release the therapeutic agents at the appropriate times and for the appropriate durations to most effectively inhibit restenosis adjacent to the ends of the stent. One skilled in the art will recognize that many combinations of therapeutic agents, therapeutic coatings, timing coatings, and positionings of the coatings are possible. Just one possibility is described below.

The outermost therapeutic coating, here therapeutic coating 222, may release a therapeutic agent including, for example, a rapamycin analog. These drugs may have antibiotic properties, stop new cells from forming, and dampen inflammation. Therapeutic coating 222 may be timed by timing coating 232 to release the rapamycin analog at an appropriate time, for example within an hour of deployment of the stent in the vessel, beginning the process of inhibiting restenosis adjacent to the ends of the stent.

Timing coating 234 may then delay release of the therapeutic agent from therapeutic coating 224 for an appropriate period of time, for example several hours after therapeutic coating 222 has finished releasing its therapeutic agent. Therapeutic coating 224 may release a superoxide dismutase mimic to break down free radicals formed as a result of basic bodily processes such as those occurring in response to injury of a vessel during deployment of a stent. Free radicals can cause additional damage to cells and tissues if not converted into less harmful products by the body's own superoxide dismutase or by a superoxide dismutase mimic. A superoxide dismutase mimic may additionally have anti-inflammatory properties.

After therapeutic coating 224 has finished releasing its therapeutic agent, timing coating 236 may delay release of the therapeutic agent from the final therapeutic coating 226 for an appropriate period of time. Therapeutic coating 226 may release a therapeutic agent such as paclitaxel, which may be most effective in inhibiting restenosis if it is released over a period of days or even months. Thus, restenosis may be inhibited for an extended period of time by this agent when released from the coating positioned nearest the stent framework.

As seen in FIG. 2, coated stent 200 additionally includes a therapeutic coating 228 disposed on the mid-portion of the stent framework. A coated stent in accordance with the present invention may, however, include coatings on only the ends of the stent framework. Therapeutic coating 228 may release a therapeutic agent that is different from the therapeutic agents released from therapeutic coatings 222, 224, and 226. It may additionally display diffusion characteristics that are different from those of coatings 222, 224, and 226. Release of the therapeutic agent from therapeutic coating 228 may be controlled by timing coating 238.

Timing coating 238 may control release of a therapeutic agent from therapeutic coating 228. The therapeutic agent included in therapeutic coating 228 may be delivered before, during, of after delivery of the therapeutic agents from the therapeutic coatings disposed on the edges of the stent.

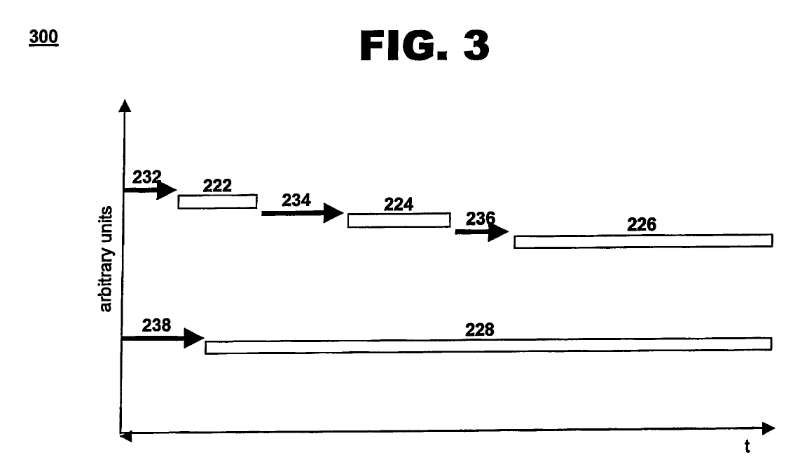

FIG. 3 shows a graphic representation of the release of therapeutic agents from the coated stent of FIG. 2.

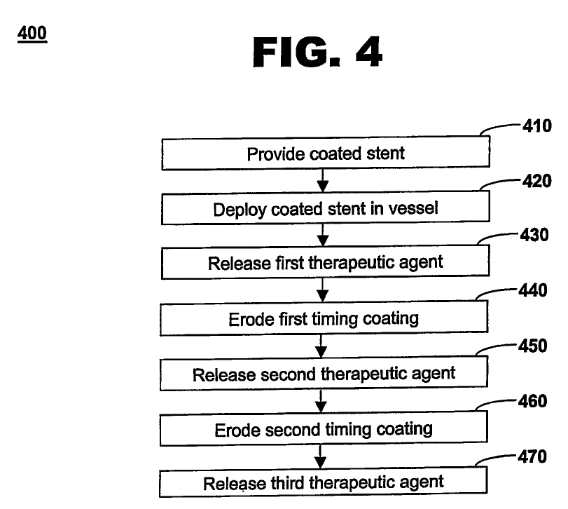

Yet another aspect of the present invention is a method of inhibiting restenosis adjacent to the ends of a stent used to treat a vascular condition. FIG. 4 shows a flow diagram of one embodiment, in accordance with the present invention at 400.

A coated stent is provided (Block 410). In this embodiment, the coated stent includes a first and second therapeutic coating and a first timing coating disposed on the distal and proximal ends of a stent framework. The first timing coating is positioned between the first and second therapeutic coatings. A third therapeutic coating and a second timing coating are disposed on a mid-portion of the stent framework, the second timing coating positioned over the third therapeutic coating.

The coated stent is deployed in a vessel (Block 420). Deployment may be accomplished by, for example, conveying the coated stent to a desired location within the vessel on a balloon catheter and inflating the balloon to deliver the stent within the vessel.

A first therapeutic agent is released from the first therapeutic coating (Block 430). The first therapeutic agent may be, for example, an antiproliferative agent, an antineoplastic agent, an antibiotic agent, an anti-inflammatory agent, a free radical scavenger, a protein, combinations thereof, and the like. More specifically, the therapeutic agent may be paclitaxel, dexamethasone, rapamycin, a rapamycin analog, a nonsteroidal anti-inflammatory drug, a steroidal anti-inflammatory drug, a superoxide dismutase mimic, apo A-1 Milano, combinations thereof, and the like.

The first timing coating is actuated (Block 440). A second therapeutic agent is released from the second therapeutic coating at a time controlled by the first timing coating (Block 450). In this embodiment, the first timing coating comprises a bioerodable polymer that erodes as a result of contact with the wall of the vessel. The timing coating is actuated when it begins to erode, and the second therapeutic agent is released after the timing coating has eroded. Alternatively, the time of release may be controlled by characteristics of the timing coating such as its thickness and permeability.

The second therapeutic agent is, preferably, different from the first therapeutic agent. The second therapeutic agent may be an antiproliferative agent, an antineoplastic agent, an antibiotic agent, an anti-inflammatory agent, a free radical scavenger, a protein, combinations thereof, and the like. More specifically, the therapeutic agent may be paclitaxel, dexamethasone, rapamycin, a rapamycin analog, a nonsteroidal anti-inflammatory drug, a steroidal anti-inflammatory drug, a superoxide dismutase mimic, apo A-1 Milano, combinations thereof, and the like.

The second timing coating, positioned on the mid-portion of the stent, is actuated (Block 450). The third therapeutic agent is released from the mid-portion of the stent at a time controlled by the second timing coating (Block 460). In this embodiment, the second timing coating comprises a bioerodable polymer that erodes as a result of contact with the wall of the vessel. The timing coating is actuated when it begins to erode, and the third therapeutic agent is released after the timing coating has eroded. Alternatively, the time of release may be controlled by characteristics of the timing coating such as its thickness and permeability.

Erosion of the second timing coating may take place simultaneously with erosion of the first timing coating. The timing coatings may, however, erode at different rates and may have different durations of erosion. Therefore, the third therapeutic agent may be released from the mid-portion of the stent before, during, or after release of the first and second therapeutic agents.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating a vascular condition, comprising:
a catheter; and
a coated stent operably coupled to the catheter, the coated stent including a plurality of therapeutic coatings disposed on a distal end and a proximal end of the stent, wherein a plurality of therapeutic agents is released from the plurality of therapeutic coatings, the therapeutic agents being released sequentially to inhibit restenosis adjacent to the ends of the stent.

2. The system of claim 1 wherein the therapeutic agents are selected from a group consisting of an antiproliferative agent, an antineoplastic agent, an antibiotic agent, an anti-inflammatory agent, a free radical scavenger, a protein, and combinations thereof.

3. The system of claim 1 wherein the therapeutic agents are selected from a group consisting of paclitaxel, dexamethasone, rapamycin, a rapamycin analog, a nonsteroidal anti-inflammatory drug, a steroidal anti-inflammatory drug, a superoxide dismutase mimic, apo A-1 Milano, and combinations thereof.

4. The system of claim 1 wherein each therapeutic coating comprises a bioerodable polymer and a therapeutic agent.

5. The system of claim 1 further comprising:
the coated stent including a plurality of timing coatings disposed on the distal and proximal ends of the stent, the timing coatings alternating with the therapeutic coatings.

6. The system of claim 5 wherein each timing coating comprises a bioerodable polymer.

7. The system of claim 1 wherein each timing coating prevents release of the therapeutic agent from the therapeutic coating positioned beneath the timing coating until a predetermined time.

8. The system of claim 1 further comprising:
the coated stent including at least one therapeutic coating disposed on a mid-portion of the stent.

9. The system of claim 7 further comprising:
at least one timing coating disposed on a mid-portion of the stent.

10. The system of claim 8 wherein the therapeutic coating disposed on the mid-portion of the stent releases a therapeutic agent that is different from the therapeutic agents released from the therapeutic coatings disposed on the distal and proximal ends of the stent.

11. The system of claim 8 wherein the therapeutic coating disposed on the mid-portion of the stent displays diffusion characteristics that are different from those of the therapeutic coatings disposed on the distal and proximal ends of the stent.

12. A coated stent, comprising:
a stent framework; and
a plurality of therapeutic coatings disposed on a distal end and a proximal end of the stent framework, wherein a plurality of therapeutic agents is released from the plurality of therapeutic coatings, the therapeutic agents being released sequentially to inhibit restenosis adjacent to the ends of the stent.

13. The coated stent of claim 12 wherein each therapeutic coating comprises a bioerodable polymer and a therapeutic agent.

14. The coated stent of claim 12 wherein the therapeutic agents are selected from a group consisting of an antiproliferative agent, an antineoplastic agent, an antibiotic agent, an anti-inflammatory agent, a free radical scavenger, a protein, and combinations thereof.

15. The coated stent of claim 12 wherein the therapeutic agents are selected from a group consisting of paclitaxel, dexamethasone, rapamycin, a rapamycin analog, a nonsteroidal anti-inflammatory drug, a steroidal anti-inflammatory drug, a superoxide dismutase mimic, apo A-1 Milano, and combinations thereof.

16. The coated stent of claim 12 further comprising:
a plurality of timing coatings disposed on the distal and proximal ends of the stent framework, the timing coatings alternating with the therapeutic coatings.

17. The coated stent of claim 16 wherein each timing coating comprises a bioerodable polymer.

18. The coated stent of claim 16 wherein each timing coating prevents release of the therapeutic agent from the therapeutic coating positioned beneath the timing coating until a predetermined time.

19. The coated stent of claim 12 further comprising:
at least one therapeutic coating disposed on a mid-portion of the stent framework.

20. The coated stent of claim 18 further comprising:
at least one timing coating disposed on a mid-portion of the stent framework.

21. The coated stent of claim 19 wherein the therapeutic coating disposed on the mid-portion of the stent releases a therapeutic agent that is different from the therapeutic agents released from the therapeutic coatings disposed on the distal and proximal ends of the stent.

22. The coated stent of claim 19 wherein the therapeutic coating disposed on the middle of the stent displays diffusion characteristics that are different from those of the therapeutic coatings disposed on the distal and proximal ends of the stent framework.

23. A method of inhibiting restenosis adjacent to the ends of a stent used to treat a vascular condition, comprising:
providing a coated stent, the coated stent including a first and a second therapeutic coating disposed on a distal and a proximal end of the stent, the first therapeutic coating including a first therapeutic agent, the second therapeutic coating including a second therapeutic agent, the coated stent further including a first timing coating positioned between the first and second therapeutic coatings;
deploying the coated stent in a vessel;
releasing the first therapeutic agent from the first therapeutic coating;
actuating the first timing coating; and
releasing the second therapeutic agent from the second therapeutic coating at a time controlled by the first timing coating.

24. The method of claim 23 wherein the therapeutic agents are selected from a group consisting of an antiproliferative agent, an antineoplastic agent, an antibiotic agent, an anti-inflammatory agent, a free radical scavenger, a protein, and combinations thereof.

25. The method of claim 23 wherein the therapeutic agents are selected from a group consisting of paclitaxel, dexamethasone, rapamycin, a rapamycin analog, a nonsteroidal anti-inflammatory drug, a steroidal anti-inflammatory drug, a superoxide dismutase mimic, apo A-1 Milano, and combinations thereof.

26. The method of claim 23 further comprising:
releasing a third therapeutic agent from a third therapeutic coating, the third therapeutic agent disposed on a mid-portion of the stent framework.

27. The method of claim 26 further comprising:
first actuating a second timing coating, the second timing coating disposed over the third therapeutic agent on a mid-portion of the stent framework.

28. The method of claim 23 wherein the second therapeutic agent is different from the first therapeutic agent.

29. The method of claim 26 wherein the third therapeutic agent is different from the first and second therapeutic agents.
